(19) United States
(12) Patent Application Publication
(10) Pub. No.: US 2010/0310628 A1
(43) Pub. Date: Dec. 9, 2010

(54) PRE-SHAPED USER-FORMABLE MICRO-MEMBRANE IMPLANTS

(75) Inventor: Erich Graf Von Waldburg-Zeil, Zurich (CH)

Correspondence Address: STOUT, UXA, BUYAN & MULLINS LLP, 4 VENTURE, SUITE 300, IRVINE, CA, 92618, US

(73) Assignee: MAST BIOSURGERY AG, Zurich (CH)

(21) Appl. No.: 12/481,311

(22) Filed: Jun. 9, 2009

Related U.S. Application Data
(63) Continuation-in-part of application No. 12/480,655, filed on Jun. 8, 2009.
Application No. 12/480,655 is a continuation-in-part of application No. 12/480,644, filed on Jun. 8, 2009.

(51) Int. Cl. A61F 2/00

(52) U.S. Cl. 424/426

(57) ABSTRACT

Precut, user-shapeable, resorbable polymer micro-membranes are disclosed. The micro-membranes are constructed of resorbable polymers, which are engineered to attenuate adhesions and to be absorbed into the body relatively slowly over time. The membranes can formed to have very thin thicknesses, for example, thicknesses between about 0.010 mm and about 0.300 mm, while maintaining adequate strength. The membranes can be extruded from polylactide polymers having a relatively high viscosity property, can be stored in sterile packages, and can be preshaped with relatively high reproducibility during implantation procedures.

### CROSS-REFERENCE TO RELATED APPLICATIONS

This application is a continuation-in-part of U.S. application Ser. No. 12/480,655, filed Jun. 8, 2009 and entitled Block-Polymer Membranes for Attenuation of Scar Tissue (Att. Docket MB8110P), is a continuation-in-part of U.S. application Ser. No. 12/480,644, filed Jun. 8, 2009 and entitled Pre-Shaped User-Formable Micro-Membrane Implants (Att. Docket MB8112P), and is related to U.S. application Ser. No. 10/385,399, filed Mar. 10, 2003 and entitled Resorbable Barrier Micro-Membranes for Attenuation of Scar Tissue During Healing (Att. Docket MA9496CON), now U.S. Pat. No. 6,673,362, the contents all of which are expressly incorporated herein by reference.

This application is also related to U.S. application Ser. No. 10/631,980, filed Jul. 31, 2003 (Att. Docket MA9604P), U.S. application Ser. No. 11/203,660, filed Aug. 12, 2005 (Att. Docket MB9828P), U.S. application Ser. No. 10/019,797, filed Jul. 26, 2002 (Att. Docket MB9962P), and U.S. application Ser. No. 12/199,760, filed Aug. 27, 2008 (Att. Docket MB8039P). The foregoing applications are commonly assigned and the entire contents of all of them are expressly incorporated herein by reference.

### BACKGROUND OF THE INVENTION

1. Field of the Invention

The present invention relates generally to medical implants and, more particularly, to resorbable membranes and methods of using the membranes.

2. Description of Related Art

Significant clinical issues relating to surgical repair of anatomical structures comprising hard or soft tissues continue to emphasize the need for one or more of (a) reducing manufacturing and distribution costs and (2) enhancing speed, simplicity, and precision of implantation, (3) while not sacrificing quality and reproducibility. In the context of surgical repair or inflammatory disease, adhesions, which can occur during the initial phases of the healing process after surgery or disease correspond to a condition which involves the formation of abnormal tissue linkages. These linkages can, for example, impair bodily function, produce infertility, obstruct the intestines and other portions of the gastrointestinal tract (bowel obstruction) and produce general discomfort, e.g. pelvic pain. The condition can in some instances be life threatening. The most common form of adhesion occurs after surgery as a result of surgical interventions, although adhesion may occur as a result of other processes or events such as pelvic inflammatory disease, mechanical injury, radiation treatment and the presence of foreign material.

Various attempts have been made to prevent postoperative adhesions. For example, the use of peritoneal lavage, heparinized solutions, procoagulants, modification of surgical techniques such as the use of microscopic or laparoscopic surgical techniques, the elimination of talc from surgical gloves, the use of smaller sutures and the use of physical barriers (membranes, gels or solutions) aiming to minimize apposition of serosal surfaces, have all been attempted. Unfortunately, very limited success has been seen with these methods. Barrier materials, in various forms such as membranes and viscous intraperitoneal solutions, which are designed to limit tissue apposition, have also met with limited success. These barrier materials can include cellulosic barriers, polytetrafluoroethylene materials, and dextran solutions.

U.S. Pat. No. 5,795,584 to Tokahura et al. discloses anti-adhesion or scar tissue reduction films or membranes, and U.S. Pat. No. 6,136,333 to Cohn et al. discloses similar structures. In the Tokahura et al. patent, a bioabsorbable polymer is copolymerized with a suitable carbonate and then formed into a non-porous single layer adhesion barrier such as a film. In the Cohn et al. patent, a polymeric hydrogel for anti-adhesion is formed without crosslinking by using urethane chemistry. Both of these patents involved relatively complex chemical formulas and/or reactions resulting in particular structures for use as surgical adhesion barriers. There continues to be a need to for an improved membrane.

### SUMMARY OF THE INVENTION

Precut, user-shapeable, resorbable polymer micro-membranes are disclosed. The micro-membranes are constructed of resorbable polymers, which are engineered to attenuate adhesions and to be absorbed into the body relatively slowly over time. The micro-membranes can formed to have very thin thicknesses, for example, thicknesses between about 0.010 mm and about 0.300 mm, while maintaining adequate strength. The micro-membranes can be extruded from polylactide polymers having a relatively high viscosity property, can be stored in sterile packages, and can be preshaped with relative speed and relatively high reproducibility during implantation procedures.

The present invention provides an improved resorbable micro-membrane that can be readily and reliably formed and positioned on, around, or in proximity to anatomical structures comprising hard or soft tissues, or implants such as disclosed in U.S. application Ser. No. 11/652,724. The micro-membrane can be used in various surgical contexts, for example, to retard or prevent tissue adhesions and reduce scarring. Furthermore, the co-polymers of the present invention may facilitate provision of relatively simple chemical reactions and/or formulations, and/or may facilitate provision of one or more of enhanced or more controllable mechanical strength and/or accelerated or more controllable degradation relative to other, e.g., mother, poly(esters).

In accordance with one exemplary implementation of the present invention a resorbable micro-membrane can be provided comprising a substantially uniform composition of a dual block copolymer. The dual block copolymer can comprise a first block that may include or consist of a polylactide and/or a polyglycolide (e.g., PLA, PGA, or PLGA) and a second block that may include or consist of a polyethylene glycol (e.g., PEG). The first block, denoted as a PLA/PGA block, may comprise a hydrophobic and biodegradable PLA/PGA block, and the second block, denoted as a PEG block, may comprise a hydrophilic PEG block.

In accordance with another feature a resorbable micro-membrane is provided comprising a substantially uniform composition of a tri block copolymer, which may comprise a first block that may include or consist of a polylactide and/or a polyglycolide (e.g., PLA, PGA, or PLGA), a second block that may include or consist of a polyethylene glycol (e.g., PEG), and a third block that may include or consist of a polylactide and/or a polyglycolide (e.g., PLA, PGA, or PLGA). The first and third blocks, each denoted as a PLA/PGA block, may comprise hydrophobic and biodegradable PLA/PGA blocks, and the second block, denoted as a PEG block, may comprise a hydrophilic PEG block.

The first PLA/PGA block and the second PEG block together may form a PLA/PGA-PEG copolymer, and addition of the third PLA/PGA block may altogether form a PLA/PGA-PEG-PLA/PGA copolymer. These copolymer micro-membranes can be formed, for example, by extrusion at, for example, an initial, relatively high viscosity property. The initially high viscosity property may facilitate reliable formation of the micro-membrane by, for example, attenuating the occurrence of, for example, breaking or tearing of the micro-membrane, during the extrusion process. After processing and sterilization, the viscosity property of the micro-membrane may typically be lower. Other viscosity properties (e.g., relatively high viscosity properties) can be used according to other aspects of the invention, in order, for example, to increase the strength of the copolymer material during the extrusion process. The extrusion process may provide the micro-membrane with a biased molecular orientation.

According to another feature, a micro-membrane has a first substantially-smooth surface and a second substantially-smooth surface, is non-porous, and is about 0.01 mm to about 0.300 mm thick as measured between the first substantially-smooth surface and the second substantially-smooth surface. The membrane thus can possess a varying cross-sectional thickness. For example, the micro-membrane can comprise at least one relatively thick portion, which can form at least a segment of an edge of the micro-membrane.

While the apparatus and method have or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112.

Any feature or combination of features described herein are included within the scope of the present invention provided that the features included in any such combination are not mutually inconsistent as will be apparent from the context, this specification, and the knowledge of one of ordinary skill in the art. In addition, any feature or combination of features may be specifically excluded from any embodiment of the present invention. For purposes of summarizing the present invention, certain aspects, advantages and novel features of the present invention are described. Of course, it is to be understood that not necessarily all such aspects, advantages or features will be embodied in any particular implementation of the present invention. Additional advantages and aspects of the present invention are apparent in the following detailed description and claims that follow.

### BRIEF DESCRIPTION OF THE DRAWINGS

FIGS. 1A and 1B illustrate a membrane structured and formed in accordance with the present invention.

### DETAILED DESCRIPTION OF THE PRESENTLY PREFERRED EMBODIMENTS

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of this disclosure, while discussing exemplary embodiments, is that the following detailed description be construed to cover all modifications, alternatives, and equivalents of the embodiments as may fall within the spirit and scope of the invention as defined by the appended claims.

Barrier micro-membranes of the present invention may be constructed from various biodegradable materials, such as resorbable polymers. In accordance with one embodiment, non-limiting polymers which may be used to form barrier micro-membranes of the present invention can include a dual block copolymer. As embodied herein, the dual block copolymer can comprise a first block that may include or consist of a polylactide and/or a polyglycolide (e.g., PLA, PGA, or PLGA) and a second block that may include or consist of a polyethylene glycol (e.g., PEG). The first block, denoted as a PLA/PGA block, can comprise a hydrophobic and biodegradable PLA/PGA block, and the second block, denoted as a PEG block, can comprise a hydrophilic PEG block. The first PLA/PGA block and the second PEG block together may form a PLA/PGA-PEG copolymer.

Other non-limiting polymers which may be used to form barrier micro-membranes of the present invention include a tri block copolymer. As embodied herein, the tri block copolymer can comprise a first block that may include or consist of a polylactide and/or a polyglycolide (e.g., PLA, PGA, or PLGA), a second block that may include or consist of a polyethylene glycol (e.g., PEG), and third block that may include or consist of a polylactide and/or a polyglycolide (e.g., PLA, PGA, or PLGA). The first block, denoted as a PLA/PGA block, can comprise a hydrophobic and biodegradable PLA/PGA block, the second block, denoted as a PEG block, can comprise a hydrophilic PEG block, and the third block, denoted as a PLA/PGA block, can comprise a hydrophobic and biodegradable PLA/PGA block. The first PLA/PGA block, the second PEG block, and the third first PLA/PGA block together may form a PLA/PGA-PEG-PLA/PGA copolymer. The micro-membranes may comprise other forms from (a) combinations and/or permutations of any one or more items disclosed or referenced herein that would be viewed by one skilled in the art to be possible or modifiable to be possible, (b) any one or more particulars, features or combinations, in whole or in part, in structure or step, disclosed or referenced in U.S. application Ser. No. 12/480,655, filed on Jun. 8, 2009 (Att. Docket MB8110P) such as starblock or 4plus block copolymers, and/or (c) combinations or permutations of (a) and (b) that would be viewed by one skilled in the art to be possible or modifiable to be possible. The entire contents of U.S. application Ser. No. 12/480,655 are incorporated herein by reference.

These copolymer micro-membranes can be formed by extrusion at an initial, relatively high viscosity property. The initially high viscosity property may facilitate reliable formation of the micro-membrane by attenuating the occurrence of, for example, breaking or tearing of the micro-membrane, during the extrusion process. After processing and sterilization, the viscosity property of the micro-membrane will typically be lower. Other relatively high viscosity properties can be used according to other aspects of the invention, in order, for example, to increase the strength of the copolymer material. The extrusion procedures advantageously can provide for efficient production of the micro-membranes. Moreover, micro-membranes which are manufactured by such extrusion techniques can be free from solvent trappings in the micro-membrane and, furthermore, can be provided with, for example, a molecular bias, including a predetermined molecular bias. Monoaxial or biaxial extrusion may be employed to manufacture the micro-membranes.

Compositions of dual block PLA/PGA-PEG copolymer, tri-block PLA/PGA-PEG-PLA/PGA copolymer, starblock copolymer, and/or 4plus block copolymer, can be extruded to form micro-membranes of the present invention. In certain embodiments, PLA/PGA-PEG copolymers taking the forms of: 1. Poly(L-lactide-co-PEG), 2. Poly(L-lactide-co-DL-lactide-co-PEG), and 3. Poly(L-lactide-co-glycolide-co-PEG); and PLA/PGA-PEG-PLA/PGA copolymers taking the forms of: 4. Poly(L-lactide-co-PEG-co-L-lactide), 5. Poly(L-lactide-co-PEG-co-L-lactide-co-DL-lactide), 6. Poly(L-lactide-co-PEG-co-L-lactide-co-glycolide), 7. Poly(L-lactide-co-DL-lactide-co-PEG-co-L-lactide-co-DL-lactide), 8. Poly(L-lactide-co-DL-lactide-co-PEG-co-L-lactide-co-glycolide), and 9. Poly(L-lactide-co-glycolide-co-PEG-co-L-lactide-co-glycolide), can be manufactured or obtained from Boehringer Ingelheim KG of Germany, for extrusion into the micro-membranes of the present invention. Exemplary synthesis and naming particulars along with examples of PLA/PGA-PEG (and/or PLA/PGA-PEG-PLA/PGA) copolymers are described in U.S. application Ser. No. 12/199,760 and U.S. application Ser. No. 12/480,655.

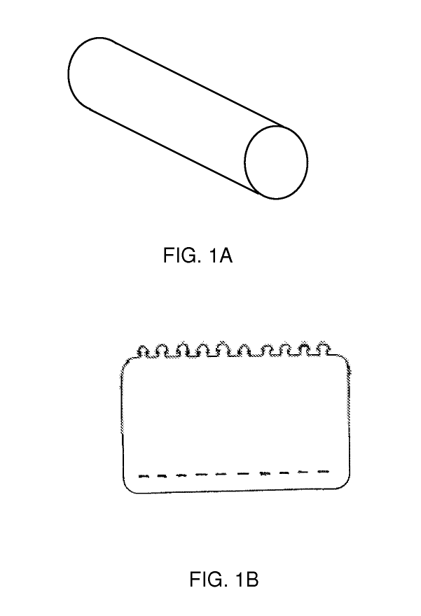

Turning to FIGS. 1A and 1B, a rolled resorbable micro-membrane is shown for implantation into a mammalian subject, such as a human. The micro-membrane can be implanted to surround part or substantially all of an anatomical structure, such as a protruding end of hard or soft tissue, or may be used, for example, for facilitating posterior lateral fusion of adjacent vertebrae such as disclosed in U.S. Pat. No. 6,719,795, the contents of which are incorporated herein by reference. The rolled resorbable micro-membrane can be sized and shaped to be placed, for instance, into contact with at least two adjacent tissues to attenuate adhesions therebetween. As presently embodied, the rolled resorbable micro-membrane comprises a first end, a second end, and an axis extending between the first end and the second end. A lumen extends along the length of the axis between the first end and the second end.

In the illustrated embodiment, the rolled resorbable micro-membrane is formed into a cylindrical shape wherein the first end and the second end are open to the lumen. This cylindrical shape may be achieved by providing a planar resorbable micro-membrane such as depicted in FIG. 1B and bringing two opposing edges together to form a configuration as depicted in FIG. 1A. In the embodiment exemplified in FIGS. 1A and 1B, a planar, rectangular resorbable micro-membrane with four edges is manipulated (e.g., folded) to bring two opposing edges of the rectangular resorbable micro-membrane into close proximity of one another. One edge can comprise tabs formed (e.g., in the shape of mushrooms) on the top edge of the micro-membrane such as shown in FIG. 1A, and another edge (e.g., an opposing edge) can comprise slots or openings such as depicted near the bottom edge of the micro-membrane in FIG. 1A. In the illustrated embodiment, each tab comprises a leading end which is rounded in shape and which transitions to a reduced-diameter neck that connects the tab to the micro-membrane, and each slot is sized for accommodating a respective leading end and neck. The two edges can be secured together by way of insertion of the tabs into the slots.

In the illustrated embodiment, the tabs have larger leading (distal) ends as compared to their proximally-disposed necks. Consistent with an objective of the invention to reduce tissue turbulence, inflammation, and/or adhesions, the tabs according to one feature can optionally or optimally be formed with rounded (e.g., mushroom) rather than pointed or barb (e.g., arrowhead) shapes, such as exemplified with the depicted semi-circular leading edge shapes. Furthermore, it may be advantageous to have a certain amount of play or flexibility in the cylindrical structure, which feature may further operate to attenuate tissue turbulence, inflammation, and/or adhesions, achievable, for example, by the width of each (e.g., one or more) tab necks being optionally or optimally formed with a smaller dimension than a width of the corresponding slot. Shapes, thicknesses, and/or areas of the tabs (e.g., one or more tabs) and/or slots (e.g., one or more slots) may vary along a length or pattern along one or more dimensions (e.g., along a length of the axis) in modified embodiments.

According to alternative implementations, one or more tab and slot pairs of the micro-membrane may alternatively or additionally be more rigidly or fixedly secured together by, for example, sutures, heat welding (discussed, infra), or staples. The resorbable micro-membranes of the present invention may comprise other perimeters besides rectangular perimeters.

Although the micro-membrane is depicted formed to have a cylindrical cross section, other cross sections, such as, for example, oval cross sections may be used. The cross sectional shapes and/or areas may vary along the length of the axis in modified embodiments. For example, a resorbable micro-membrane may be rolled to have a slightly conical or hour glass shape. Moreover, the resorbable micro-membrane may be rolled around another object or, alternatively, may be rolled without the use of any forming structure.

For relatively thick implementations (e.g., thicknesses greater than about 500 microns) the resorbable micro-membrane can be brought to its glass transition temperature either before or after being formed or shaped on (e.g., wrapped around) an object (e.g., a mandrel) and, subsequently, allowed to cool while still formed on the object. After the resorbable micro-membrane has cooled to a temperature below the glass transition temperature, the shaped resorbable micro-membrane can be removed from the object to thereby yield a formed resorbable micro-membrane having at least a portion in the shape or resembling the shape (e.g., cylindrical shape) of the object. As an example, the resorbable micro-membrane may be placed into a heated saline solution, rolled, with the tabs either before or after such insertion being inserted into the slots, and, subsequently, lifted out of the heated saline solution and allowed to cool in the formed configuration.

In the illustrated embodiment of FIGS. 1A and 1B, the two opposing edges of the resorbable micro-membrane are brought together (e.g., around a mandrel) to form a seam. The flexible connection between the tab necks and slots facilitates, enhances or adds flexibility to the rolled resorbable micro-membrane, so that, for example, the rolled resorbable micro-membrane may be shaped, with or without heating, to have a rectangular, oval, triangular or other cross section. For example, a mandrel with a rounded-corner triangular shape may be used to shape a planar sheet to have an approximately triangular cross section. One or both of the two opposing edges of the resorbable micro-membrane may need to be trimmed so that a smooth seam is generated. In modified embodiments, the seam may comprise a slight or substantial overlap of one of the two edges of the resorbable micro-membrane over the other edge. This overlap may span a length, for example, which is equal to a radius or even a diameter of the resorbable micro-membrane. In other words, the overlap may span an arc length of between 1 and approximately 180 degrees. In modified embodiments, the overlap may be even greater. The overlapping edge may completely encircle the resorbable micro-membrane one or more times for, as just one example, added strength. In modified embodiments, the opposing edges may not contact one another at all, so that a gap is formed therebetween. In such an embodiment, the tabs may comprise elongate shapes (e.g., elongate necks) to bridge the gap and fit within the slots. The gap can be relatively small, spanning an arch length of about 1 to about 45 degrees and, more preferably, an arch length of about 1 to 5 degrees. Preferably, the gap should not be so large as to impede the function of the resorbable micro-membrane of, in typical implementations, attenuating adhesions.

The amount of overlap, and any gap size, may vary along the length of the axis. For example, gaps may be formed at certain locations at the seam, and/or overlaps may be formed between the gaps for reinforcement. Various means for attaching the rolled resorbable micro-membrane to tissue or implant structures are contemplated. For example, the rolled resorbable micro-membrane can be secured via frictional engagement alone. Portions of the rolled resorbable micro-membrane may be secured to tissue or implant material using resorbable bone screws or tacks. Tucking or folding of portions of the rolled resorbable micro-membrane into anatomical crevices or about tissue or implant elements may be sufficient to fix its position in other embodiments. An adhesive such as a fibrin sealant, or a resorbable cyanoacrylate adhesive may further be utilized to secure the rolled resorbable micro-membranes, alone or in combination with the above means of attachment.

In accordance with one aspect of the present invention, one or more portions of the rolled resorbable micro-membrane can be heat bonded, such as with a bipolar electro-cautery device, ultrasonically welded, or similarly sealed directly to one or more tissue or implant elements. Such a device can be used to heat the barrier micro-membrane at various locations, such as at the edges and at points therebetween, at least above its glass transition temperature, and preferably above its softening point temperature. The glass transition temperature of an exemplary material (70:30 poly L-lactide-co-D, L-lactide (PLDLA)) is about 55 degrees Celsius, while its softening (e.g., melting) point temperature is much above that. The material can be heated along with adjacent tissue or implant material such that the two components bond together at their interface. In another embodiment, the rolled resorbable micro-membrane can be heat bonded or sealed directly to itself such as, for example, at the seam, and/or to muscle or other adjacent hard tissue, soft tissue or implant material. The term “implant” is intended to include, among other things, any structure disclosed or referenced in U.S. patent application Ser. No. 11/652,724, the entire contents of which are incorporated herein by reference. For example, the rolled resorbable micro-membrane may be formed into a cylinder in vitro, or wrapped around a tissue or implant element in vivo, and then heat joined to itself Moreover, the technique of heat-sealing the rolled resorbable micro-membrane material to itself and/or to body tissue or implant material may be combined with another attachment method for enhanced anchoring. For example, the rolled resorbable micro-membrane material may be temporarily affixed in position using two or more points of heat sealing (i.e., heat welding) using an electro-cautery device, and sutures, staples or glue can then be added to secure the barrier micro-membrane into place. The seam may them be heat welded to itself or, alternatively, formed to slightly overlap itself without any heat welding at the seam for added flexibility of the rolled resorbable micro-membrane.

The base material of the rolled resorbable micro-membrane can be configured to be rigid enough to maintain an available space, e.g., lumen, within the rolled resorbable micro-membrane along a length of the rolled resorbable micro-membrane under its own weight without collapsing. In the illustrated embodiment, an available space, for growth, expansion, or just movement of tissue, can in some implementations be maintained within the rolled resorbable micro-membrane along a length of the rolled resorbable micro-membrane. Additionally, the base material is resorbable, according to the presently preferred embodiment. The micro-membrane can be porous to one or more of vessels, cells, and liquids, or, alternatively, non-cell permeable pores may be used or no pores altogether, in which case cells and vasculature could still proliferate into the inner space of the rolled resorbable micro-membrane through the opposing open ends of the rolled resorbable micro-membrane.

As presently embodied, the rolled resorbable micro-membrane comprises either a biodegradable synthetic material or a biodegradable natural material, or both. The biodegradable synthetic material may comprise polymers, for example, and the biodegradable natural material may comprise collagen, for example. A thickness of the base material can range, for example, between about 10 microns and about 300 microns, and in other implementations can range from about 0.25 mm and 3 mm, such as, for example, between 0.5 mm and 2 mm. The base material of the rolled resorbable micro-membrane may be configured with greater or smaller thicknesses in modified embodiments. The ranges of base material thickness and other micro-membrane features discussed herein are preferably implemented by the present invention in order to optimize the rolled resorbable micro-membrane to different environmental conditions. Examples of the different environmental conditions encountered in different applications include the location, shape, composition, type, size, and condition of adjacent hard or soft tissues and/or implants.

The combination of the rolled resorbable micro-membrane and the fixation device may in some instances be constructed for operating together to relieve stress shielding within the protected space of the rolled resorbable micro-membrane. For example, the fixation device may be installed with a slight looseness, may be constructed to be fully or partially resorbable, may be removed from the patient at a suitable time, or may be configured of a resorbable or partially resorbable material.

A micro-membrane of the present invention can have at least one substantially smooth-surface. Preferably, a micro-membrane of the present invention has two (opposing) substantially smooth surfaces. As measured between the opposing surfaces, a micro-membrane of the present membrane can have a thickness of about 0.01 mm to about 0.3 mm and, more preferably, about 0.01 mm to about 0.1 mm. In a preferred embodiment, a micro-membrane of the present invention has a thickness of about 0.015 mm to about 0.025 mm. In another preferred embodiment, a micro-membrane of the present invention has a thickness of about 0.02 mm. The micro-membranes of the present invention can be formed to have thicknesses greater than about 0.3 mm, such as thicknesses from 0.3 mm to about 2 or 3 mm, for example, in modified embodiments.

A preferred micro-membrane of the present invention can comprise a substantially uniform composition of copolymer. The copolymer can have a biased molecular orientation in the micro-membrane as a consequence, for example, of extrusion.

As used herein, the term “non-porous” refers to a material which is generally water tight and, in accordance with a preferred embodiment, not fluid permeable. However, in a modified embodiment of the invention micro-pores (i.e., fluid permeable but not cell permeable) may exist in the micro-membrane of the present invention, to the extent, for example, that they do not substantially disrupt the smoothness of the surfaces of the resorbable micro-membrane to cause scarring of tissue. In substantially modified embodiments for certain applications, pores which are cell permeable but not vessel permeable may be manufactured and used.

As presently embodied, many of the thinner micro-membrane thicknesses can be sufficiently contoured even in the absence of heating to glass transition temperature. As presently embodied, the resorption of the rolled resorbable micro-membrane can be between approximately 2 and 24 months. In one embodiment, micro-membranes of the present invention can be capable of resorbing into the mammalian body within a period, for example, of about 18 to about 24 months from an initial implantation of the micro-membrane into the mammalian body. The rolled resorbable micro-membrane can be resorbed within the body of the patient to a point where substantial strength is no longer present within a period of approximately 1 year. Complete resorption of the rolled resorbable micro-membrane may subsequently occur after a total period of 1.5 to 2 years has elapsed since the initial implantation. In other embodiments, the rolled resorbable micro-membrane may comprise in whole or part non-resorbable plastic or metallic materials.

The micro-membranes may be used in a number of surgical applications, including: surgical repair of fracture orbital floors, surgical repair of the nasal septum and perforated ear drum micro-membrane, as a protective sheathing to facilitate osteogenesis, surgical repair of the urethral anatomy and repair of urethral strictures, prevention of synostosis in completed corrective surgery for cranial fusions and forearm fractures, lessening of soft-tissue fibrosis or bony growth, as a temporary covering for prenatal rupture omphalocele during staged repair procedures, guided tissue regeneration between the teeth and gingival margin, tympanic membrane repairs, dual coverings and neural repair, heart vessel repair, hernia repair, tendon anastomoses, temporary joint spacers, wound dressings, scar coverings, and as a covering for gastroschisis. The micro-membrane of the present invention can be particularly suitable for preventing tissue from abnormally fibrotically joining together following surgery, which can lead to abnormal scarring and/or interfere with normal physiological functioning. In some cases, such scarring can force and/or interfere with follow-up, corrective, or other surgical operations.

The very thin construction of these micro-membranes is believed to substantially accelerate the rate of absorption of the micro-membranes, compared to rates of absorption of thicker micro-membrane implants of the same material. It is believed, however, that resorption into the body too quickly of the micro-membrane may, in some instances, yield undesirable drops in local pH levels, thus introducing/elevating, for example, local inflammation, discomfort and/or foreign antibody responses. Further, a resulting uneven (e.g., cracked, broken, roughened or flaked) surface of a micro-membrane degrading too early may undesirably cause tissue turbulence between the tissues before, for example, adequate healing has occurred, potentially resulting in tissue inflammation and/or scarring. In other instances, a different (e.g., more rapid) resorption may be desired in one or more areas of a patient, and/or at one or more points in time of one or more surgical procedures, so that, in accordance with an aspect of the present invention, rates of absorption may be controlled or varied, temporally and/or spatially, by varying the materials of the micro-membrane or parts thereof

Micro-membranes in accordance with an aspect of the present invention may be provided in rectangular shapes that are for example several centimeters on each side, or can be cut and formed into other specific shapes, configurations and sizes, by the manufacturer before packaging and sterilization. According to a feature of the present invention, they preferably take the shape depicted in FIGS. 1A and 1B. In modified embodiments, various known formulations and copolymers of, for example, polylactides may affect the physical properties of the micro-membrane. The micro-membranes of the present invention may be sufficiently flexible to conform over and/or around anatomical structures, although some heating in a hot water bath may be necessary for thicker configurations. In modified embodiments, certain polylactides which may become somewhat more rigid and brittle at thicknesses above, for example, 0.25 mm and which may be softened by formation with other polymers, copolymers and/or other monomers, e.g., epsilon-caprolactone, for example, may be implemented to form micro-membranes.

Moreover, in accordance with another aspect of the present invention, the micro-membrane may comprises a substance for cellular control, such as at least one of a chemotactic substance for influencing cell-migration, an inhibitory substance for influencing cell-migration, a mitogenic growth factor for influencing cell proliferation and a growth factor for influencing cell differentiation. Such substances may be impregnated in the micro-membrane, but may also be coated on one or more surfaces of the micro-membrane. In addition, substances may be contained in discrete units on or in the micro-membrane, which may be effective to facilitate selective release of the substances when the micro-membrane is inserted into a patient. Other configurations for accommodating different anatomical structures may be formed. For example, configurations may be designed to be formed into, for example, cone structures to fit around base portions with protrusions extending through the centers of the micro-membranes. Suture perforations may be formed around perimeters of the micro-membranes, and cell and vessel permeable pores may be included as well.

In general, any particulars, features or combinations thereof (in whole or in part, in structure or step), described or referenced herein, may be combined with any particulars, features or combinations thereof (in whole or in part, in structure or step), described or referenced in any of the documents mentioned herein, including without limitation U.S. application Ser. No. 11/203,660 and U.S. application Ser. No. 12/199,760 (in whole or in part, in structure or step, provided that the particulars or features included in any such combination are not mutually inconsistent.

In accordance with one implementation of the present invention, the pre-formed micro-membranes can be preformed and sealed in sterilized packages for subsequent use by the surgeon. Since one objective of the micro-membranes of the present invention can be to reduce sharp edges and surfaces, preformation of the micro-membranes is believed to help, in some instances, facilitate, albeit to a relatively small degree, rounding of the edges for less rubbing, tissue turbulence and inflammation. That is, the surfaces and any sharp edges of the micro-membranes are believed to be capable of ever so slightly potentially degrading over time in response to exposure of the micro-membranes to moisture in the air, to thereby form rounder edges. This is believed to be an extremely minor effect. Moreover, any initial heating to glass temperature of the pre-cut micro-membranes just before implanting may conceivably further round any sharp edges. Furthermore, the very micro-membranes of the present invention may be particularly susceptible, at least theoretically, to these phenomena, and, perhaps to a more noticeable extent, are susceptible to tearing or damage from handling, thus rendering the pre-forming of the micro-membranes potentially beneficial for preserving the integrity thereof

In accordance with an aspect of the present invention, a surgical prosthesis (e.g., a resorbable scar-tissue reduction micro-membrane system) can comprise an adhesion-resistant region (e.g., a biodegradable region, a biodegradable side, a membrane and/or a micro-membrane) of copolymer composition as described herein, and further may comprise an optional tissue-ingrowth region (e.g., another membrane, a bridging membrane, a biodegradable region and/or a biodegradable side or mesh) which may or may not comprise, for example, a copolymer composition as described herein.

The surgical prosthesis (e.g., biodegradable surgical prosthesis) can be constructed for use in the repair of soft tissue defects, such as soft tissue defects resulting from incisional and other hernias and soft tissue defects resulting from extirpative tumor surgery. The surgical prosthesis may also be used in cancer surgeries, such as surgeries involving sarcoma of the extremities where saving a limb is a goal. Other applications of the surgical prosthesis of the present invention may include laparoscopic or standard hernia repair in the groin area, umbilical hernia repair, paracolostomy hernia repair, femora hernia repair, lumbar hernia repair, and the repair of other abdominal wall defects, thoracic wall defects and diaphragmatic hernias and defects.

According to an aspect of the present invention, the tissue-ingrowth region and the adhesion-resistant region may differ in both (A) surface appearance and (B) surface function. For example, the tissue-ingrowth region can be constructed with at least one of a surface topography (appearance) and a surface composition (function), either of which may facilitate strength, longevity or lack thereof, and/or a substantial fibroblastic reaction in the host tissue relative to for example the anti-adhesion region. On the other hand, the adhesion-resistant region can be constructed with at least one of a surface topography and a surface composition, either of which may facilitate, relative to the tissue-ingrowth region, an anti-adhesive effect between the biodegradable surgical implant and host tissues.

### A. Surface Topography (Appearance):

The tissue-ingrowth region can be formed to have an open, non-smooth and/or featured surface comprising, for example, alveoli and/or pores distributed regularly or irregularly. In further embodiments, the tissue-ingrowth region can be formed to have, additionally or alternatively, an uneven (e.g., cracked, broken, roughened or flaked) surface which, as with the above-described surfaces, may cause tissue turbulence (e.g., potential tissue inflammation and/or scarring) between host tissues and the tissue-ingrowth region.

Over time, with respect to the tissue-ingrowth region, the patient's fibrous and collagenous tissue may substantially completely overgrow the tissue-ingrowth region, growing over and affixing the tissue-ingrowth region to the tissue. In one implementation, the tissue-ingrowth region comprises a plurality of alveoli or apertures visible to the naked eye, through or over which the host tissue can grow and achieve substantial fixation.

As an example, pores may be formed into the tissue-ingrowth region by punching or otherwise machining, or by using laser energy. Non-smooth surfaces may be formed, for example, by abrading the tissue-ingrowth region with a relatively course surface (e.g., having a 40 or, preferably, higher grit sandpaper-like surface) or, alternatively, non-smooth surfaces may be generated by bringing the tissue-ingrowth region up to its softening or melting temperature and imprinting it with a template (to use the same example, a sandpaper-like surface). The imprinting may occur, for example, during an initial formation process or at a subsequent time.

On the other hand, the adhesion-resistant region can be formed to have a closed, continuous, smooth and/or non-porous surface. In an illustrative embodiment, at least a portion of the adhesion-resistant region is smooth comprising no protuberances, alveoli or vessel-permeable pores, so as to attenuate occurrences of adhesions between the tissue-ingrowth region and host tissues.

In a molding embodiment, one side of the press may be formed to generate any of the tissue-ingrowth region surfaces discussed above and the other side of the press may be formed to generate an adhesion-resistant region surface as discussed above. Additional features (e.g., roughening or forming apertures) may subsequently be added to further define the surface of, for example, the tissue-ingrowth region. In an extrusion embodiment, one side of the output orifice may be formed (e.g. ribbed) to generate a tissue-ingrowth region (wherein subsequent processing can further define the surface such as by adding transverse ribs/features and/or alveoli) and the other side of the orifice may be formed to generate an adhesion-resistant biodegradation region surface. In one embodiment, the adhesion-resistant region is extruded to have a smooth surface and in another embodiment the adhesion-resistant region is further processed (e.g., smoothed) after being extruded.

### B. Surface Composition (Function):

As presently embodied, the tissue-ingrowth region comprises a first material, and the adhesion-resistant region comprises a second material which is different from the first material. In modified embodiments, the tissue-ingrowth region and the adhesion-resistant region may comprise the same or substantially the same materials. In other embodiments, the tissue-ingrowth region and the adhesion-resistant region may comprise different materials resulting from, for example, an additive having been introduced to at least one of the tissue-ingrowth region and the adhesion-resistant region.

According to an implementation of the present invention, the adhesion-resistant region is constructed to minimize an occurrence of adhesions of host tissues (e.g., internal body viscera) to the surgical prosthesis. In modified embodiments, the adhesion-resistant region and the tissue-ingrowth region of the surgical prosthesis may be formed of the same material or relatively less divergent materials, functionally speaking, and the adhesion-resistant region may be used in conjunction with an anti-inflammatory gel agent applied, for example, onto the adhesion-resistant region at a time of implantation of the surgical prosthesis. According to other broad embodiments, the adhesion-resistant region and the tissue-ingrowth region may be formed of any materials or combinations of materials disclosed herein (including embodiments wherein the two regions share the same layer of material) or their substantial equivalents, and the adhesion-resistant region may be used in conjunction with an anti-inflammatory gel agent applied, for example, onto the adhesion-resistant region at a time of implantation of the surgical prosthesis.

The tissue-ingrowth region can be formed of similar and/or different materials to those set forth above, to facilitate strength, longevity or lack thereof, and/or direct post-surgical cell colonization via, for example, invoking a substantial fibroblastic reaction in the host tissue. In an illustrated embodiment, the tissue-ingrowth region is constructed to be substantially incorporated into the host tissue and/or to substantially increases the structural integrity of the surgical prosthesis. Following implantation of the surgical prosthesis, body tissues (e.g., subcutaneous tissue and/or the exterior fascia) commence to incorporate themselves into the tissue-ingrowth region. While not wishing to be limited, it is believed that the body, upon sensing the presence of the tissue-ingrowth region of the present invention, is disposed to send out fibrous tissue which grows in, around and/or through and at least partially entwines itself with the tissue-ingrowth region. In this manner, the surgical prosthesis can become securely attached to the host body tissue.

Regarding different materials, according to an aspect of the present invention, the tissue-ingrowth region can comprise a biodegradable (e.g., resorbable) polymer composition having one or more different characteristics than that or those of a biodegradable (e.g., resorbable) polymer composition of the adhesion-resistant region. The different characteristics may include (1a) time or rate of biodegradation affected by additives, (1b) time or rate of biodegradation affected by polymer structures/compositions, (2) polymer composition affecting strength or structural integrity, and (3) ability to facilitate fibroblastic reaction.

In accordance with a method of the present invention, the surgical prosthesis can be used to facilitate repair of, for example, a hernia in the ventral region of a body. An implanted surgical prosthesis having both an adhesion-resistant region disposed on one side and having a tissue-ingrowth region disposed on a second side of the surgical prosthesis can be provided. The abdominal wall can include muscle enclosed and held in place by an exterior fascia and an interior fascia. An interior layer, called the peritoneum, can cover the interior side of the interior fascia. The peritoneum is a softer, more pliable layer of tissue that forms a sack-like enclosure for the intestines and other internal viscera. A layer of skin and a layer of subcutaneous fat cover the exterior fascia.

Surgical repair of a soft tissue defect (e.g., a hernia) can be performed by using, for example, conventional techniques or advanced laparoscopic methods to close substantially all of a soft tissue defect. According to one implementation, an incision can be made through the skin and subcutaneous fat, after which the skin and fat can be peeled back followed by any protruding internal viscera (not shown) being positioned internal to the hernia. In certain implementations, an incision can be made in the peritoneum followed by insertion of the surgical prosthesis into the hernia opening so that the surgical prosthesis is centrally located in the hernia opening. One or both the tissue-ingrowth region and the adhesion-resistant region may be attached by, e.g., suturing to the same layer of the abdominal wall, e.g., the relatively-strong exterior fascia. Alternatively, the adhesion-resistant region may be attached to another member, such as the interior fascia and/or the peritoneum. The tissue-ingrowth region can be surgically attached to the exterior fascia while the adhesion-resistant region can be attached to the tissue-ingrowth region and/or optionally to the exterior fascia using, e.g., heat bonding, suturing, and/or other affixation protocols disclosed herein or their substantial equivalents. Those possessing skill in the art will recognize that other methods of sizing/modifying/orientating/attaching a surgical prosthesis of this invention may be implemented according to the context of the particular surgical procedure.

The size of the surgical prosthesis typically will be determined by the size of the defect. Use of the surgical prosthesis in a tension-free closure may be associated with less pain and less incidence of post surgical fluid accumulation. Exemplary sutures may be implemented to at least partially secure the surgical prosthesis to the abdominal wall structure. The sutures can be implemented so that no lateral tension is exerted on the exterior fascia and/or muscle. When disrupted, the skin and fat may be returned to their normal positions, with for example the incisional edges of the skin and fat being secured to one another using suitable means such as subsurface sutures.

In modified embodiments of the present invention, one or both of the tissue-ingrowth region and the adhesion-resistant region of the surgical prosthesis, can be heat bonded (or in a modified embodiment, otherwise attached, such as by suturing). Heat bonding may be achieved, for example, with a bipolar electro-cautery device, ultrasonicly welding, or similar sealing between the tissue-ingrowth region and the adhesion-resistant region and/or directly to surrounding tissues. Such a device can be used to heat the surgical prosthesis at various locations, such as at edges and/or at points in the middle, at least above its glass transition temperature, and preferably above its softening point temperature. The material is heated, e.g., along with adjacent tissue, such that the two components bond together at their interface. The heat bonding may also be used initially, for example, to secure the tissue-ingrowth region to the adhesion-resistant region. Since the tissue-ingrowth region serves more of a load-bearing function, a few typical embodiments may exclude heat-bonding as the sole means for securing this region to host tissues. In other embodiments, the technique of heat bonding the surgical prosthesis to itself or body tissue may be combined with another attachment method for enhanced anchoring. For example, the surgical prosthesis may be temporarily affixed in position using two or more points of heat bonding using an electro-cautery device, and sutures, staples or glue can subsequently (or in other embodiments, alternatively) be added to secure the surgical prosthesis into place.

The tissue-ingrowth region and the adhesion-resistant region may be arranged to form more than one layer or substantially one layer, or the regions may both belong to a single, integrally formed layer. For example, the tissue-ingrowth region and the opposing adhesion-resistant region may be arranged in two layers, wherein one of the regions is disposed on top of, and opposite to, the other region.

In one embodiment, the tissue-ingrowth region and the adhesion-resistant region may be combined on a single side of the surgical prosthesis in, for example, substantially one layer, wherein the regions are adjacent each other on one side of the surgical prosthesis. As a slight deviation, a surgical prosthesis having a tissue-ingrowth region on at least one (and preferably, both) side(s) thereof may be manufactured using any of the techniques described herein and, subsequently, an adhesion-resistant region may be formed on, e.g., one side, by smoothing, filling, or otherwise processing an area of the tissue-ingrowth region with a suitable material as disclosed herein or technique (e.g., coating or filling with a liquid or flowable polymer composition, and/or mechanically smoothing) to thereby form an adhesion-resistant region having adhesion-resistant properties relative to those of the tissue-ingrowth region.

Similarly, a patch of adhesion-resistant region may be sized and affixed (e.g., heat bonded, such as with a bipolar electro-cautery device, ultrasonicly welded, or similarly affixed) at a time of implantation directly to at least one of the tissue-ingrowth region and surrounding host tissues. In modified embodiments, the affixing may be accomplished using, for example, press or adhesive bonding, or sutures. In further embodiments, at least part of the affixing may occur at a time of manufacture of the surgical prosthesis before packaging. The patch of adhesion-resistant region alternatively may be partially affixed (e.g., using techniques enumerated in this paragraph) at, for example, a non-perimeter or central area thereof to an area (e.g., a non-perimeter or central area) of the tissue-ingrowth region, so that a surgeon can trim the adhesion-resistant region (and/or the tissue-ingrowth region) at a time of implantation while the adhesion-resistant biodegradable implant is affixed to the tissue-ingrowth region. For instance, a tissue-ingrowth region may substantially surround an adhesion-resistant region on one side of the surgical prosthesis, and only a tissue-ingrowth region may be formed on the other side of the surgical prosthesis. In such an implementation, the adhesion-resistant region of the surgical prosthesis can be sized and shaped so as to substantially cover any opening created by the soft tissue defect, with the tissue-ingrowth regions facilitating surgical attachment to, and incorporation into, the host tissue on at least one side of, and, preferably, on both sides of, the surgical prosthesis.

In modified embodiments, the tissue-ingrowth region and/or the adhesion-resistant region on a given surface or surfaces of the surgical prosthesis each may be of any size or shape suited to fit the particular soft tissue defect. For example, either of the tissue-ingrowth region and/or the adhesion-resistant region on a given surface of the surgical prosthesis may have shapes of ovals, rectangles and various complex or other shapes wherein, for each such implementation, the two regions may have essentially the same, or different, proportions and/or dimensions relative to one another.

In general, various techniques may be employed to produce the surgical prosthesis, which typically has one or two layers defining the tissue-ingrowth region and the adhesion-resistant region. Useful techniques include solvent evaporation methods, phase separation methods, interfacial methods, extrusion methods, molding methods, injection molding methods, heat press methods and the like as known to those skilled in the art. The tissue-ingrowth region and the adhesion-resistant region may comprise two distinct layers or may be integrally formed together as one layer.

The tissue-ingrowth region and the adhesion-resistant region may be partially or substantially entirely formed or joined together. Joining can be achieved by mechanical methods, such as by suturing or by the use of metal clips, for example, hemoclips, or by other methods, such as chemical or heat bonding.

The above-described embodiments have been provided by way of example, and the present invention is not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. As iterated above, any feature or combination of features described and referenced herein are included within the scope of the present invention provided that the features included in any such combination are not mutually inconsistent as will be apparent from the context, this specification, and the knowledge of one of ordinary skill in the art. For example, any of the implants and implant components, sub-components, or uses, and any particulars or features thereof, or other features, including method steps and techniques, may be used with any other structure and process described or referenced herein, in whole or in part, in any combination or permutation. Accordingly, the present invention is not intended to be limited by the disclosed embodiments, but is to be defined by reference to the appended claims.

What is claimed is:

1. A resorbable scar-tissue reduction micro-membrane system for attenuating a formation of post-surgical scar tissue between a healing post-surgical site and adjacent surrounding tissue following an in vivo surgical procedure on the post-surgical site, the system having a pre-implant configuration, which is defined as a configuration of the system immediately before the system is formed between the post-surgical site and the adjacent surrounding tissue, the system comprising a substantially planar membrane of resorbable polymer base material having a first substantially-smooth side and a second substantially-smooth side, the substantially planar membrane of resorbable polymer base material comprising a single layer of resorbable polymer base material between the first substantially-smooth side and the second substantially-smooth side, the single layer of resorbable polymer base material including a tab with a leading end which is rounded in shape and which transitions to a reduced-diameter neck that connects the tab to the membrane and further including a slot for accommodating the leading end and the neck, wherein the single layer of resorbable polymer base material consists essentially of a tri block copolymer including a first hydrophobic block of one or more of a lactide and a glycolide, a second hydrophilic block of a polyethylene glycol, and a third hydrophobic block of one or more of a lactide and a glycolide.

2. The resorbable scar-tissue reduction micro-membrane system as set forth in claim 1, wherein the single layer of resorbable polymer base material is cut to have a non-rectangular and non-circular shape and to have a size and shape suitable for snugly and anatomically fitting over an anatomic structure to thereby attenuate formation of scar tissue between the anatomic structure and surrounding tissue, and is sealed in a sterile packaging.

3. The resorbable scar-tissue reduction micro-membrane as set forth in claim 1, wherein the single layer of resorbable polymer base material is cut with tabs to be folded over and around an anatomic structure and is sealed in a sterile packaging.

4. The resorbable scar-tissue reduction micro-membrane system as set forth in claim 1, wherein the thickness is about 100 microns.

5. The resorbable scar-tissue reduction micro-membrane system as set forth in claim 1, wherein the thickness is about 200 microns.

6. The resorbable scar-tissue reduction micro-membrane system as set forth in claim 1, wherein the single layer of resorbable polymer base material is not fluid permeable.

7. The resorbable scar-tissue reduction micro-membrane system as set forth in claim 1, wherein the single layer of resorbable polymer base material is impregnated with at least one of a chemotactic substance for influencing cell-migration, an inhibitory substance for influencing cell-migration, a mitogenic growth factor for influencing cell proliferation, a growth factor for influencing cell differentiation, and factors which promote neoangiogenesis.

8. The resorbable scar-tissue reduction micro-membrane system as set forth in claim 7, wherein the resorbable scar-tissue reduction micro-membrane system is sealed in a sterile packaging.

9. A resorbable scar-tissue reduction membrane system for attenuating a formation of post-surgical scar tissue between a healing post-surgical site and adjacent surrounding tissue following an in vivo surgical procedure on the post-surgical site, the system having a pre-implant configuration, which is defined as a configuration of the system immediately before the system is formed between the post-surgical site and the adjacent surrounding tissue, the system comprising:
a substantially planar membrane of resorbable polymer base material having a first substantially-smooth side and a second substantially-smooth side, the substantially planar membrane of resorbable polymer base material comprising a layer of resorbable polymer base material between the first substantially-smooth side and the second substantially-smooth side, the layer of resorbable polymer base material having a substantially uniform composition;
wherein a thickness of the layer of resorbable polymer base material, measured between the first substantially-smooth side and the second substantially-smooth side, is between about 10 microns and about 300 microns;
wherein the layer of resorbable polymer base material is non-porous;
wherein the substantially planar membrane of resorbable polymer base material includes a tab with a leading end which is rounded in shape and which transitions to a reduced-diameter neck that connects the tab to the membrane and further includes a slot for accommodating the leading end and the neck; and
wherein the substantially planar membrane of resorbable polymer base material is disposed in a package.

10. The resorbable scar-tissue reduction micro-membrane system as set forth in claim 9, wherein the single layer of resorbable polymer base material consists essentially of a dual block copolymer including a first hydrophobic block of one or more of a lactide and a glycolide and a second hydrophilic block of a polyethylene glycol.

11. The resorbable scar-tissue reduction micro-membrane system as set forth in claim 9, wherein the single layer of resorbable polymer base material consists essentially of a tri block copolymer including a first hydrophobic block of one or more of a lactide and a glycolide, a second hydrophilic block of a polyethylene glycol, and a third hydrophobic block of one or more of a lactide and a glycolide.

12. The resorbable scar-tissue reduction micro-membrane system as set forth in claim 9, wherein the thickness is about 100 microns.

13. The resorbable scar-tissue reduction micro-membrane system as set forth in claim 9, wherein the single layer of resorbable polymer base material is cut to have a non-rectangular and non-circular shape, is cut to anatomically fit over and protect an exiting nerve root, and is sealed in a sterile packaging.

14. The resorbable scar-tissue reduction micro-membrane system as set forth in claim 9, wherein the single layer of resorbable polymer base material is cut to have a non-rectangular and non-circular shape, is cut with tabs to be folded over and around and to protect an exiting nerve root, and is sealed in a sterile packaging.

15. The resorbable scar-tissue reduction micro-membrane system as set forth in claim 9, wherein the single layer of resorbable polymer base material is cut to have a non-rectangular and non-circular shape and is sealed in a sterile packaging.

16. The resorbable scar-tissue reduction micro-membrane system as set forth in claim 9, the substantially planar membrane of resorbable polymer base material comprising a single layer of resorbable polymer base material between the first substantially-smooth side and the second substantially-smooth side, the single layer of resorbable polymer base material having a substantially uniform composition.

17. The resorbable scar-tissue reduction micro-membrane system as set forth in claim 9, wherein the resorbable scar-tissue reduction micro-membrane system further includes another membrane, which comprises a thickness less than 2000 microns and which is permeable.

18. The resorbable scar-tissue reduction micro-membrane system as set forth in claim 17, wherein the other membrane is a bridging membrane that is cell permeable.

19. The resorbable scar-tissue reduction micro-membrane system as set forth in claim 17, wherein the other membrane is vessel permeable.

20. The resorbable scar-tissue reduction micro-membrane system as set forth in claim 17, wherein the other membrane comprises a thickness between 500 microns and 2000 microns.
